(19) United States
(12) Patent Application Publication
(10) Pub. No.: US 2024/0408245 A1
(43) Pub. Date: Dec. 12, 2024

(54) PREFILLED CUTANEOUS PATCH WITH LANDMARK

(71) Applicant: All Skin Inc, Calgary (CA)

(72) Inventors: Ryan T. Lewinson, Calgary (CA); Isabelle Vallerand, Calgary (CA); John-Douglas Matthew Hughes, Calgary (CA)

(73) Assignee: All Skin Inc, Calgary, AB (CA)

(21) Appl. No.: 18/706,563

(22) PCT Filed: Nov. 1, 2022
(86) PCT No.: PCT/CA2022/051615

§ 371 Date: May 1, 2024

Related U.S. Application Data
(60) Provisional application No. 63/274,176, filed on Nov. 1, 2021.

(51) Int. Cl. A61K 49/00; A61K 9/70

(52) U.S. Cl. CPC A61K 49/0006; A61K 9/7084

(57) ABSTRACT

A prefilled cutaneous patch is provided for testing for allergic contact dermatitis and/or skin sensitivity. The patch comprises one or more wells containing ingredients that may be mixed with a suitable base (e.g., petrolatum, an aqueous solution, an anhydrous compound). The patch further comprises a backing that features an adhesive surface for adhering to a user's skin, and one or more landmarks that facilitate marking of the skin to visually demonstrate an orientation of the applied patch (e.g., which end is up, locations of wells). A landmark may comprise a dye or ink, which may be installed in one or more wells or elsewhere on the patch such that it is transferred to the user's skin when the patch is applied. Also, or alternatively, a landmark may comprise a stencil through which the user may mark the skin with a pattern that promotes interpretation of the results of the testing.

### RELATED APPLICATION(S)

This application claims priority under 35 U.S.C. § 119 to U.S. Provisional Patent Application No. 63/274,176, entitled “Pre-Filled Patch Test Chamber and Method for Orientation Landmarking of Cutaneous Patches”, which was filed Nov. 1, 2021 and is incorporated herein by reference.

### BACKGROUND

This disclosure relates to the field of medicine. More particularly, a cutaneous patch apparatus is provided with methods of using the apparatus.

For quite some time, patch-based testing has been the standard diagnostic test for allergic contact dermatitis (or skin allergy or skin sensitivity), which is a skin condition caused by a hypersensitivity reaction to a chemical or biological allergen. For example, patches may be used to find the cause of an allergic reaction in a person. Similarly, evaluation of a cosmetic or cosmetic-related product may involve applying a selected substance to an area of skin and determining its effect upon skin parameters such as comedogenicity, dryness, and redness.

Currently, however, the use of patch-based testing requires multiple visits to one's medical professional. Typically, during a first visit a physician applies one or more patches to the patient's skin after the patches are manually prepared in the physician's office with selected allergens. Approximately two days later, the patches are removed during a second visit. A few days later the patient makes another visit, at which time the physician evaluates skin reactions (if there are any).

This protocol has several drawbacks. First, it can be time-consuming for the physician (or physician's staff) to manually prepare each patch with the desired allergens, which may have limited shelf-lives. Second, the configuration of typical patches prevents them from being prepared in advance: for example, moisture may wick through a surface of a patch and degrade (or spoil) an allergen before the patch is used. Third, the physician or physician's staff must trace, on a patient's skin (e.g., with semi-permanent or ultraviolet ink), outlines of the patches and/or allergen compartments in order to aid subsequent identification and analysis of the test results. Fourth, the patient's activity may be limited while wearing a patch, to avoid making the patch wet and/or obliterating the patch outlines. These requirements, and/or others, prevent a traditional patch-based method of allergy testing from being conducted via telehealth and/or with other modern digital technologies.

Therefore, what is needed is an apparatus and corresponding methods for performing patch-based allergy and/or skin sensitivity testing free from the constraints and restrictions associated with traditional patch-based testing.

### SUMMARY

In some embodiments, apparatus and methods are provided for facilitating or performing patch-based testing for allergic contact dermatitis (or skin allergy) or other skin sensitivity without repeat visits to a medical professional.

In these embodiments, a cutaneous patch apparatus is configured with one or more wells or chambers that contain allergens or other ingredients, which may be mixed with petrolatum or another substance. The patch also comprises an adhesive surface (e.g., surrounding the well(s) on an obverse side) for adhering the patch to a user's skin, and one or more transferable landmarks that mark the person's skin when the patch is affixed. A reverse side or surface of the patch may be waterproof or water-resistant, thereby allowing the user to maintain normal activities (e.g., bathing, exercising) without degrading the diagnostic value of the patch, and may be capable of being printed upon (e.g., with instructions, with identifying marks).

In some embodiments, a user receives a prefilled patch from a medical professional, retailer, or via mail, removes a liner that protects the allergen/ingredient wells and the adhesive, and places the patch in contact with his or her skin to allow it to adhere. After a specified time period, the user removes and discards the patch and, possibly after another delay, may photograph the patch's former location. A provider of the patch may provide an app to facilitate image capture, submission of the image for analysis, and/or analysis of the image to identify possible allergic and/or sensitive skin reactions, or the user may manually upload the image for analysis. The image may be analyzed by a medical professional and/or a suitably trained machine-learning model.

The patch includes one or more landmarks that, while in contact with the user's skin, cause the skin to be marked with information (e.g., symbols) to help determine the location and/or orientation of the patch. For example, the landmarks may assist a diagnostician in identifying the locations of individual wells and/or the allergens/ingredients contained in one or more wells. A landmark may comprise a temporary tattoo or a pattern of dye (or ink or other marking agent) that is transferred to the user's skin and that will generally be visible for multiple days. In some alternative embodiments, a landmark may comprise one or more unique stencils that, while the patch is affixed to the user's skin, allow the user to mark the stencil's pattern upon his or her skin using a marker or similar implement.

### DESCRIPTION OF THE FIGURES

FIG. 1 is a diagram depicting a patch for testing for allergic contact dermatitis or other skin sensitivity, in accordance with some embodiments.

FIGS. 2A-C are diagrams of patches for testing for allergic contact dermatitis or other skin sensitivity while providing landmarking, in accordance with some embodiments.

FIGS. 3A-B are diagrams of the reverse and obverse sides of a patch with one or more landmarks, in accordance with some embodiments.

FIG. 4 is a flow chart illustrating a method of constructing a cutaneous patch with one or more landmarks, in accordance with some embodiments.

FIG. 5 is a flow chart demonstrating a method of testing for allergic contact dermatitis or other skin sensitivity with a prefilled cutaneous patch, in accordance with some embodiments.

FIGS. 6A-D illustrate automated processing of an image of a user's skin after a patch is removed, in order to determine the orientation of the patch and its wells, in accordance with some embodiments.

### DETAILED DESCRIPTION

The following description is presented to enable any person skilled in the art to make and use the disclosed embodiments, and is provided in the context of one or more particular applications and their requirements. Various modifications to the disclosed embodiments will be readily apparent to those skilled in the art, and the general principles defined herein may be applied to other embodiments and applications without departing from the scope of those that are disclosed. Thus, the present invention or inventions are not intended to be limited to the embodiments shown, but rather are to be accorded the widest scope consistent with the disclosure.

In some embodiments, a cutaneous patch having one or more wells or chambers prefilled with ingredients is provided for testing for allergic contact dermatitis or other skin sensitivity (collectively referred to herein as “skin sensitivity”). The ingredients included with a patch may include allergens, cosmetic products, components of cosmetic products, and/or other substances, which are hereafter referred to simply as “ingredients.” The patch is suitable for application and removal by a person (e.g., a user, a patient) without assistance from a medical professional. The patch includes one or more landmarks (e.g., temporary tattoos, ink or dye patterns, stencils) for marking (or facilitating marking) of the person's skin to reveal the location and/or orientation of the patch and/or individual wells.

In some implementations, a patch is at least partially constructed of water-resistant or waterproof material to prevent moisture from entering the wells and also to prevent leakage of the ingredients, which may be mixed with a non-reactive base (e.g., petrolatum, an aqueous or anhydrous substance) before or after being added to separate wells. Wells may include foam frames to further prevent leakage. In addition, an adjunct layer may be provided for covering the patch and surrounding skin when additional moisture protection is desired or recommended.

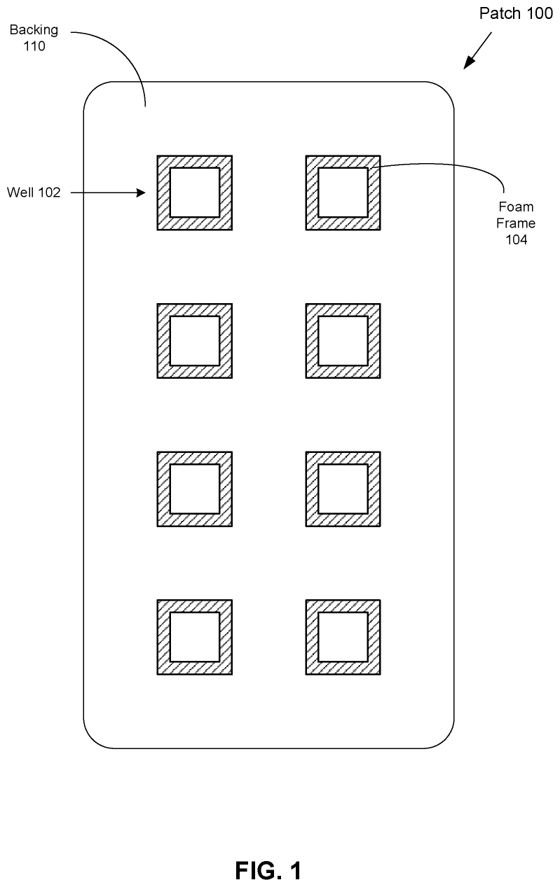

FIG. 1 is a diagram of a prefilled cutaneous patch for testing for skin sensitivity, in accordance with some embodiments. In these embodiments, patch 100 comprises multiple (eight) wells or compartments 102 formed in, on or upon backing 110, but in other embodiments a patch may contain any number of wells (i.e., one or more). Similarly, patches and individual wells of a patch are not limited to rectangular or square forms. Wells 102 in FIG. 1 are empty, but are in condition for receiving ingredients.

In some implementations of the illustrated embodiment, each well measures approximately 1 cm by 1 cm, including optional foam frame 104, and is surrounded on all sides by approximately 1 cm of backing 110. In these implementations, patch 100 measures approximately 5 cm wide by 9 cm tall.

The illustrated side (e.g., the obverse side) of backing 110 and patch 100 may be adhesive over the entire surface or may be adhesive over only part of the surface (e.g., everywhere except wells 102). Therefore, the illustrated side of backing 110 is the side that will be placed against a user's skin and features sufficient adhesive surface area to prevent being accidentally dislodged after installation, and also to resist incursion by water and other liquids. An individual well 102 need not be completely filled with an ingredient, but will contain sufficient quantity of the ingredient to ensure it is in contact with a user's skin when affixed to the user.

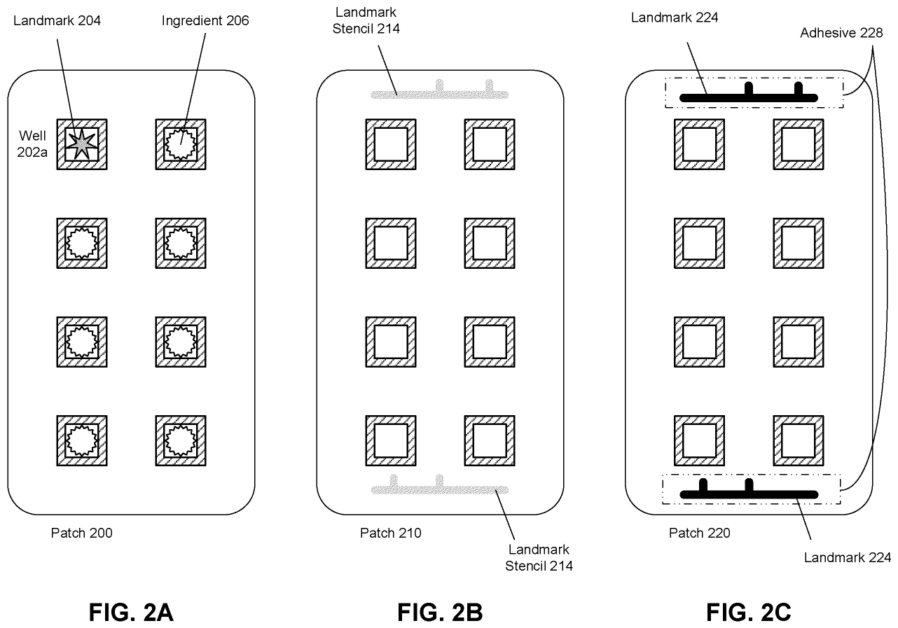

FIGS. 2A-C are diagrams of patches for testing for skin sensitivity while providing landmarking, in accordance with some embodiments. Each figure illustrates a separate type of landmarking, and it may be noted that a cutaneous patch provided herein may feature multiple landmarks of the same or different types.

Patch 200 of FIG. 2A features one or more wells (e.g., well 202a) containing landmark 204 in the form of dye (or ink) that, when the patch is affixed to a user's skin, will transfer to and mark the skin. The dye may or may not be hypoallergenic and may be or may include paraphenylenediamine (PPD), Henna, Jagua, disperse blue, etc. Other wells of patch 200 contain ingredients 206.

Patch 210 of FIG. 2B features landmark stencils or templates 214 that a user may use to mark his or her skin after the patch is applied. More specifically, each stencil 214 comprises a slot, bore, or other cut-out through which a user may mark the underlying skin with some type of marking agent (e.g., ink, dye, a marker).

Patch 220 of FIG. 2C features transferrable landmarks 224 that will mark the user's skin after the patch is affixed to the skin. Landmarks 224 may contain any dye, ink, or other marking agent safe for human use. In some implementations, transferrable landmarks 224 comprise Jagua-based temporary tattoos, may include optional adhesive 228 to adhere the tattoos to the user's skin, and may further include temporary covers (not shown in FIG. 2C) that are removed prior to placement of patch 220 in contact with a user's skin.

It may be noted that a landmark (e.g., landmark 204, landmark stencils 214, landmarks 224) may be configured to aid a diagnostician's determination of the orientation of the corresponding patch. For example, in FIGS. 2B and 2C the landmark stencils and landmarks include horizontal bars that completely or substantially extend across the width of horizontally adjacent wells. In addition, landmark stencils 214 and landmarks 224 feature short perpendicular extensions, which are oriented in the same direction in both stencils or landmarks to allow the diagnostician to identify a reference end of the patch (e.g., a “top” end). Further, in the illustrated embodiments, the short extensions identify key axes of the patch, such as a vertical midline and centers of each column of wells. Even if a mirror image is provided (with details reversed across the vertical axis), reference to landmarks 224 and tracings made through landmark stencils 214 will allow the diagnostician to discern the original or ‘correct’ left and right sides of the patch, and thereby correctly interpret the test results. In some embodiments, a trained machine-learning model may perform some or all of the functions of a diagnostician.

Landmark 204 also aids in determining the orientation of patch 200, after it is removed, based on the fact that the landmark may always be placed in a specific well, such as the top left well, the bottom right well, etc.

In other embodiments, a landmark may be configured differently from what is illustrated in FIGS. 2A-C but generally will promote rapid identification of where ingredients contacted the user's skin, by identifying one or more locations or boundaries of wells, a centroid of the patch, etc. For example, landmarks may be employed on the long sides of the patch in addition to, or instead of, the short sides (as shown in FIGS. 2B-C). As another example, in embodiments in which a single patch may be cut or otherwise separated into multiple smaller patches, landmarks may accompany individual wells (e.g., to identify their location and/or contents), rather than (or in addition to) revealing the orientation of the patch when it was affixed to a user's skin.

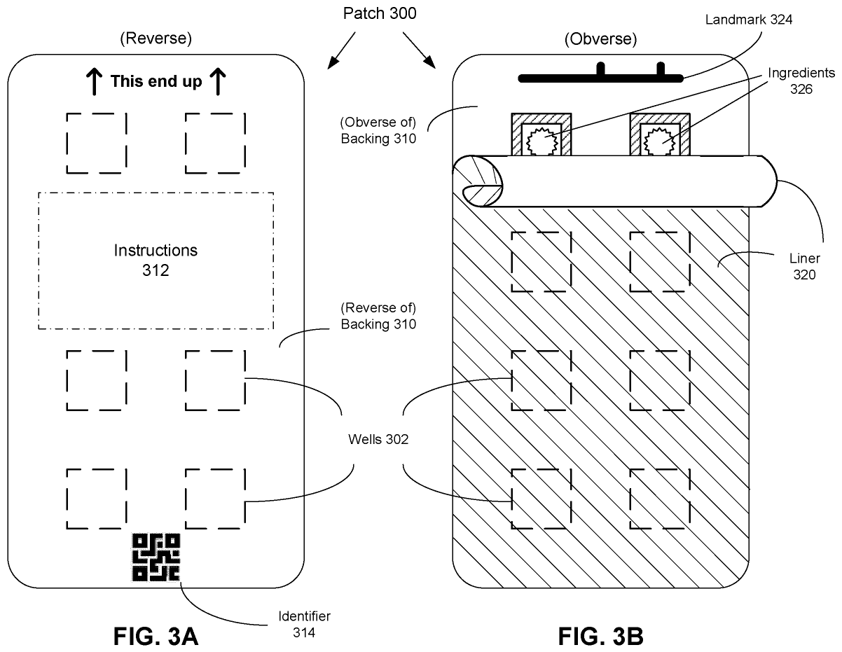

FIGS. 3A-B illustrate the reverse and obverse sides of a patch for testing for skin sensitivity, in accordance with some embodiments. In these embodiments, the obverse side of cutaneous patch 300 is the side that will adhere to a user's skin (e.g., after removing a liner or protective cover), while the reverse side of the patch is the side that will be visible after the patch is applied. In both figures, outlines of wells 302 (which cannot be viewed until the patch is prepared for use) appear as dashed squares or rectangles.

The reverse of patch 300 and backing 310, as shown in FIG. 3A, may include orientation information (e.g., “This end up”), instructions 312 for preparing and applying the patch, identifier 314 (e.g., a QR (Quick Response) code, a barcode), and/or other information (e.g., a logo, brand name, artwork). In some implementations, however, the reverse side of the patch and the backing may be substantially blank or primarily feature an aesthetic appearance that may be pleasing to a user, and may be of any color.

The obverse of patch 300 and backing 310, as shown in FIG. 3B, is initially covered by liner 320, which protects the adhesive surface of the backing and prevents ingredients 326 from being dislodged. Liner 320 is partially rolled back or down to show further detail. The obverse of backing 310 features one or more landmarks 324 for marking the user's skin and assisting with later interpretation or analysis of the results of the skin sensitivity testing.

In different embodiments, a cutaneous patch or constituent components of a cutaneous patch (e.g., backing, wells, liner) may be constructed from different materials suitable for medical use. For example, in some embodiments, a medical foam such as Avery-Dennison MED 5695R single-coated foam may be formed into moisture-resistant wells (e.g., approximately 0.6 mm in depth) via manual cutting, die cutting, or some other process.

In some other embodiments, wells may be defined by foam frames, as depicted in FIGS. 1 and 2A-C, on a suitable backing material. In these other embodiments, suitable backing materials include silicone, cellulose, hydrocolloid, or some other (bio) polymer. Specific commercial products that may be used as backing material include 3M™ single-sided polyethylene medical tape 1503 and 3M single-sided elastic nonwoven fabric medical tape 9907T/9907 W/9907HTW, both of which are printable, thereby facilitating custom designs on the reverse side of a patch. It may be noted that one or more of these products feature peelable liners that are water-resistant and that can be used as the liner for finished patches (e.g., liner 320 of FIG. 3B). The foam frames may be constructed from polyethylene, ethylene vinyl acetate, silicone, polyurethane, sponge, hydrocolloid, cellulose, or an engineered (bio) polymer. In some implementations, however, a backing and its wells may be additively manufactured (e.g., with a biodegradable material).

After wells are formed for a patch, such as by forming depressions in a foam backing or by forming foam frames on a backing, individual wells may be partially or fully filled with a mix of petrolatum (or other suitable base) and a selected ingredient. In some implementations, wells are filled to less than their maximum capacity in order to allow sealing of the wells without loss or mixing of ingredients, while ensuring eventual transfer of the ingredients to users' skin. In these implementations, while a patch is affixed to a user's skin, the user may depress individual wells in order to ensure effective contact of well ingredients with the skin.

A patch may be sealed by applying a liner (e.g., liner 320 of FIG. 3B), which may be done within a negative pressure environment (e.g., a vacuum) to further prevent mixing of ingredients from different wells of the patch. After being sealed, a patch may be enclosed within a sleeve or envelope (e.g., made of aluminum foil, plastic, or a suitable biodegradable material) that features resistance to heat and moisture in order to promote extended shelf life.

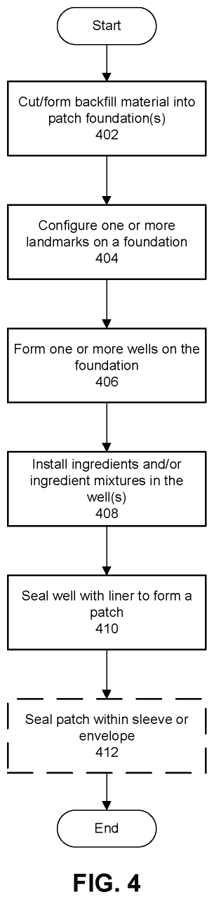

FIG. 4 is a flowchart demonstrating a method of creating a prefilled cutaneous patch in accordance with some embodiments. One or more of the operations may be omitted, repeated, and/or performed in a different order. Accordingly, the specific arrangement of steps shown in FIG. 4 should not be construed as limiting the scope of the embodiments.

In operation 402, a backfill or base material made of a pliable foam (such as one of those identified above) is cut into one or more pieces to serve as foundations for individual patches. One side of the backfill and each resulting foundation is adhesive and will be the obverse side of a patch, while the reverse side may be printable in addition to being waterproof or water-resistant. Instructions, identifying markings, aesthetic designs, and/or other content may be printed on the reverse side prior to complete assembly of the patch.

A foundation for a 2×4 patch illustrated in FIG. 1 (i.e., a patch having eight wells arranged in a rectangular pattern) may be approximately 5 cm wide by 9 cm tall, with wells approximately 1 cm by 1 cm in dimension. In contrast, a foundation for a 1×1 patch (i.e., a patch having a single well) may be approximately 3 cm by 3 cm in size. Corners of each foundation may be rounded to help resist undesired peeling of the finished patch. Rounding of corners may be performed during operation 402 or after a patch is assembled, at which time a liner that seals the patch may also be trimmed or shaped to match the foundation.

In operation 404, one or more landmarks and/or landmark stencils are applied to a foundation. As discussed above, a landmark may comprise a temporary tattoo or strategically shaped and placed dye or ink that will transfer to a user's skin. A stencil may be formed by cutting or otherwise excising a portion of the foundation (completely through the foundation) to allow a user to mark his or her skin after the finished patch is applied to the skin. In embodiments that employ one or more well-based landmarks, each of which comprises a collection of dye, ink or other marking substance within a well, such landmarks may be configured later in the process of manufacturing a patch (i.e., after the wells are formed or constructed), such as part of operation 408. In embodiments in which a foundation is created for a 1×1 patch (i.e., a patch having a single well), landmarks may or may not be added. There will be at most one reaction to the well's contents, which would be readily identifiable, and if no reaction occurs it is unnecessary to pinpoint where the ingredient contacted the user's skin.

In some implementations, a landmark in the form of a temporary tattoo is printed directly onto the foundation. In other implementations, temporary tattoo landmarks are separate components and are affixed to the foundation. A temporary tattoo may or may not have an additional cover or liner (separate from a liner that seals an entire obverse side of a patch), which is removed prior to application of a patch to a user's skin.

In operation 406, foam frames may be applied (e.g., adhered to) a foundation to define individual wells of a patch. A rectangular or square frame comprises four sides (similar to a picture frame), with an open top and an open or closed bottom. In other words, the top of the frame is open to allow deposition of an ingredient and to allow it to contact a user's skin, but the bottom of the frame may be defined by additional foam framing or may be open, in which case the ingredient will reside in contact with the underlying foundation. Wells are not limited to square and/or rectangular forms. A foam frame may adhere to the adhesive surface of the foundation and/or be affixed with additional adhesive. In some embodiments, each side of a frame may be approximately 10 mm tall, 2.5 mm wide, and 0.6 mm thick.

In some implementations, frames are partially embedded in the backing material to create a moat around the well, thereby helping prevent leakage. In other implementations in which wells are constructed contiguously with the backing material (e.g., as pits or depressions in the backing), there may be no frame.

In operation 408, each of one or more wells formed on the foundation is populated with a mix of one or more ingredients and a base, such as petroleum jelly, an aqueous solution, or an anhydrous compound. For example, each mixture may comprise approximately 80% petroleum jelly and 20% ingredient (by volume), although the ratio may vary considerably from one embodiment to another. Different wells of the foundation may be populated with different ingredients and/or multiple wells may feature a common ingredient (e.g., in different concentrations). Similarly, one or more wells may contain a mixture of multiple ingredients. As indicated above, one or more wells may receive a dye or other marking substance to serve as landmarks.

In operation 410, a waterproof or water-resistant liner is placed over the obverse side of the foundation to seal the wells and complete assembly of a patch. Pressure may be applied as necessary to form a strong seal that will resist leakage of well contents. This operation may be performed in a vacuum or partial vacuum to help prevent ingredient leakage.

In optional operation 412, the patch may be further sealed within a sleeve, envelope or other enclosure that will enhance the shelf life of the patch by resisting the effects of moisture and/or heat. Enclosing the patch in this manner may also prepare the patch for transportation.

In other embodiments, instead of framing wells (e.g., with foam frames), a backfill of suitable thickness is employed from which wells may be formed by excavation or coring to a depth and width sufficient to contain an effective volume of an ingredient or ingredient/petrolatum mixture. For example, wells may be approximately 0.6 mm deep.

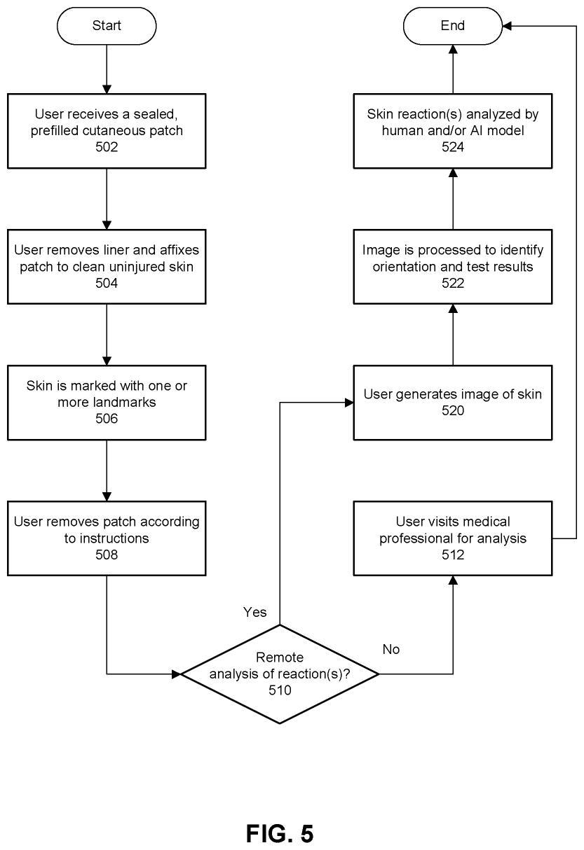

FIG. 5 is a flowchart demonstrating a method of performing skin sensitivity testing with a prefilled cutaneous patch, in accordance with some embodiments. One or more of the indicated operations may be omitted, repeated, and/or performed in a different order. Accordingly, the specific arrangement of steps shown in FIG. 5 should not be construed as limiting the scope of the embodiments.

In operation 502, a user (or patient) receives a sealed, prefilled cutaneous patch comprising one or more wells or chambers that contain an ingredient, possibly in the form of a mixture that also includes petroleum jelly.

In operation 504, the user removes any outer wrapping (e.g., a sleeve, pouch, or other enclosure) and removes a liner, thereby exposing the one or more wells of an obverse side of the patch. In some implementations, the user will also remove separate liners or covers that protect one or more landmarks. The user affixes the patch to clean and uninjured skin in accordance with instructions that accompany the patch and/or were provided by a physician or other medical professional. Adhesive on the obverse side of the patch promotes a secure seal of the patch against the skin, thereby preventing any ingredient from escaping its well and/or mixing with any other ingredient.

In operation 506, while the patch is in place, the user's skin is marked with one or more landmarks that were incorporated into the patch. As discussed previously, for example, the obverse side of the patch may feature one or more temporary tattoos, patterns of dye or other marking agent (possibly within a well), and/or stencils. If the patch includes any landmark stencils, the user may assist in the marking process by writing through the stencil with a marker or similar implement.

In operation 508, based on the instructions received by the user, he or she removes the patch after a suitable time period (e.g., 48 hours). The patch may be discarded.

In operation 510, if the user is going to visit a medical professional to obtain an immediate analysis of the results of the allergy testing, the method continues at operation 512 with the user visiting that professional to receive the analysis (e.g., and diagnosis). The professional's analysis/diagnosis of the results may be augmented by an artificial intelligence (AI) model for efficiency and for digitally capturing a record of the testing.

Otherwise, in operation 520, the user takes a photograph or otherwise obtains an image of the skin area that was in contact with the patch. In some embodiments, for example, a source or distributor of the patch may provide an app that executes on a device of the user and that facilitates capture of the image.

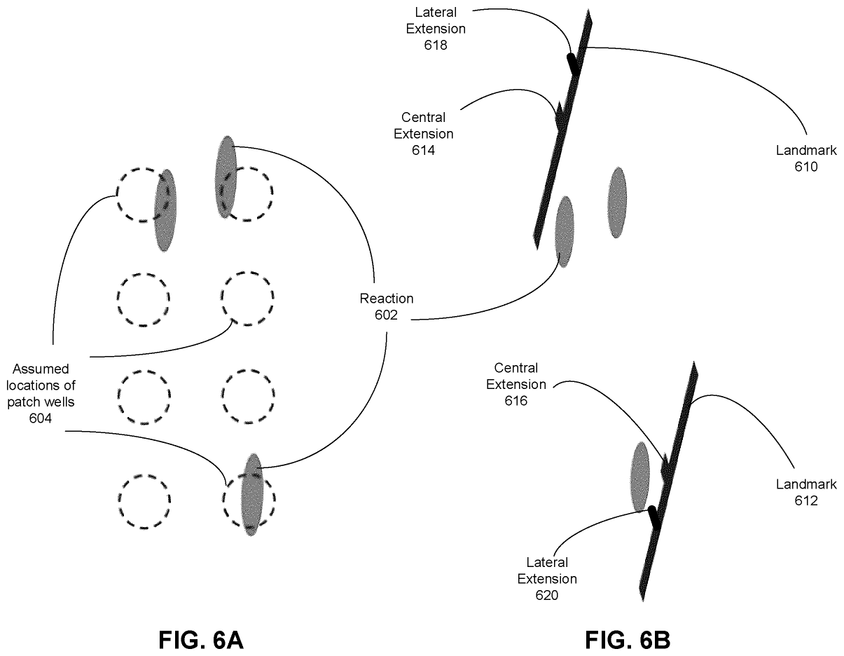

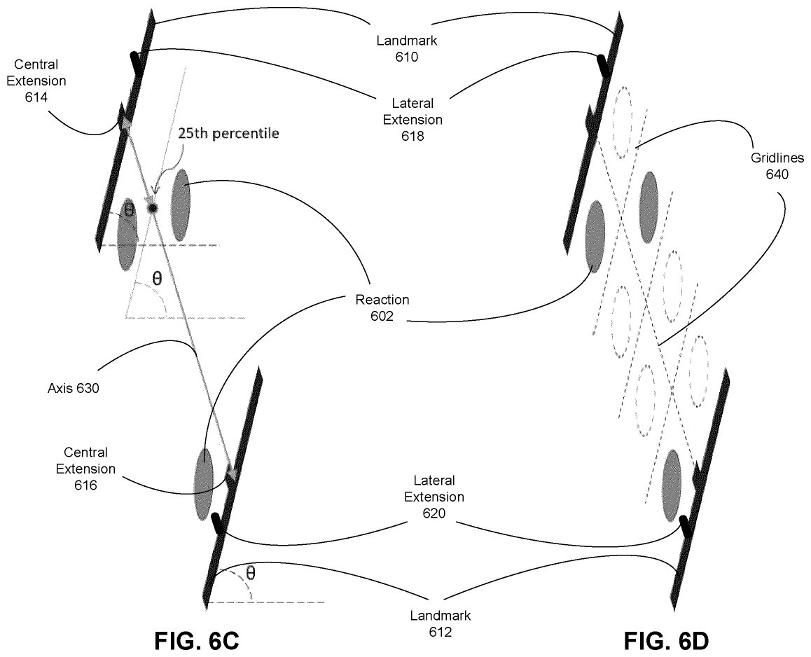

In these embodiments, the location and/or configuration of one or more landmarks as captured in the image may assist in the manual determination and/or automated determination (e.g., by a trained machine-learning model) of the orientation of the patch and/or locations of one or areas of interaction between the skin and ingredients contained in the patch, as discussed below in conjunction with FIGS. 6A-D.

In operation 522, the image is processed to determine correct orientation and to ensure the areas of contact between the skin and the ingredient(s) are captured. For example, an app may recognize the landmarks transferred to the user's skin in order to automatically guide the user to manually orient the photo correctly regardless of whether the skin demonstrates any reaction to any of the ingredients.

In operation 524, the photo is analyzed by a human and/or a machine-learning model to diagnose the test results.

In some embodiments, a cutaneous patch is provided that features one or more landmarks but no ingredients (or wells for ingredients). In these embodiments, a user may apply the patch in order to mark his or her skin with a grid or other visual reference(s) created by the landmarks, and then applies desired ingredients and/or other substances within the grid. By recording the locations of the substances (e.g., using the visual references of the landmark(s)), in the case of a reaction the user can easily determine the offending substance. In yet other embodiments, a user may obtain a patch comprising landmarks and empty wells in which the user may place desired ingredients.

FIGS. 6A-D depict the machine-based processing of an image of skin reactions caused by a prefilled cutaneous patch, in accordance with some embodiments. In particular, these figures show how the image may be interpreted to correctly identify the orientation of the reactions that result from interaction of the patch's ingredients with a user's skin, in order to map them to corresponding wells of the patch.

FIG. 6A demonstrates how parallax may affect a naïve interpretation of a photograph of skin sensitivity testing. In particular, three reactions 602 have occurred after use of a 2×4 patch and are visibly evident on the user's skin (as solid ovals). When the photograph is taken from a plane that is not the same as, or parallel with, the plane of the patch that was applied, an initial interpretation of the reactions may be as shown, wherein the dashed circles indicate the assumed locations 604 of the patch's wells. As shown below, this interpretation is incorrect.

FIG. 6B illustrates the same skin reactions 602, but augmented with landmarks transferred from a patch. In particular, landmarks 610, 612 bracket the skin area that was covered by the patch, such as at ‘top’ and ‘bottom’ ends of the patch. It may be noted that central extensions 614, 616 allow identification of a central (e.g., vertical) axis of the patch and the affected skin area, while lateral extensions 618, 620 identify axes of the two columns of wells (one on each side of the vertical axis).

FIG. 6C illustrates further processing of the image of the skin reactions of FIGS. 6A-B in order to identify the layout of the wells of the patch that caused the reactions. In these embodiments, imaginary or virtual axis 630 is calculated as a line or line segment connecting central extensions 614, 616. In addition, because the configuration of the patch is known (i.e., 2×4), it is known that four pairs of wells straddling the central axis are arrayed between the landmarks with equal or approximately equal distancing (e.g., as shown in FIG. 1), along axes corresponding to lateral extensions 618, 620. Therefore, by virtually demarcating distances that are approximately 25%, 50% and 75% of the distance along axis 630 from one extension or landmark to the other, the skin area encompassed by the patch can be virtually divided into eight reaction areas, each of which corresponds to one well of the patch. Additional processing and mathematical modifications can be made in the case of extreme parallax, such as when the relative size of a landmark (e.g., in comparison to the size of another landmark) is much different from reality or what may be expected to be seen in an accurate image. In some situations, the nature of the image may frustrate processing and lead to a request to the user to retake the photograph.

FIG. 6D illustrates the final division of the affected skin area into separate areas corresponding the wells of the patch that covered the area. Imaginary or virtual gridlines 640 are determined by the program or model that processes the image of the skin reactions. Thus, it can be seen that computer-aided interpretation of an image, with appropriate landmarking, can help ensure that skin reactions (if any) are associated with the correct ingredients.

An environment in which one or more embodiments described above are executed may incorporate a general-purpose computer or a special-purpose device such as a hand-held computer or communication device. Some details of such devices (e.g., processor, memory, data storage, display) may be omitted for the sake of clarity. A component such as a processor or memory to which one or more tasks or functions are attributed may be a general component temporarily configured to perform the specified task or function, or may be a specific component manufactured to perform the task or function. The term “processor” as used herein refers to one or more electronic circuits, devices, chips, processing cores and/or other components configured to process data and/or computer program code.

Data structures and program code described in this detailed description are typically stored on a non-transitory computer-readable storage medium, which may be any device or medium that can store code and/or data for use by a computer system. Non-transitory computer-readable storage media include, but are not limited to, volatile memory; non-volatile memory: electrical, magnetic, and optical storage devices such as disk drives, magnetic tape, CDs (compact discs) and DVDs (digital versatile discs or digital video discs), solid-state drives, and/or other non-transitory computer-readable media now known or later developed.

Methods and processes described in the detailed description can be embodied as code and/or data, which may be stored in a non-transitory computer-readable storage medium as described above. When a processor or computer system reads and executes the code and manipulates the data stored on the medium, the processor or computer system performs the methods and processes embodied as code and data structures and stored within the medium.

Furthermore, the methods and processes may be programmed into hardware modules such as, but not limited to, application-specific integrated circuit (ASIC) chips, field-programmable gate arrays (FPGAs), and other programmable-logic devices now known or hereafter developed. When such a hardware module is activated, it performs the methods and processes included within the module.

The foregoing embodiments have been presented for purposes of illustration and description only. They are not intended to be exhaustive or to limit this disclosure to the forms disclosed. Accordingly, many modifications and variations will be apparent to practitioners skilled in the art. The scope is defined by the appended claims, not the preceding disclosure.

What is claimed is:

1. A cutaneous skin patch, comprising:
a backing having an adhesive surface that adheres to skin of a human user;
one or more wells containing ingredients; and
one or more landmarks.

2. The cutaneous skin patch of claim 1, wherein application of the patch to the skin causes the skin to be marked with the one or more landmarks.

3. The cutaneous skin patch of claim 2, wherein application of the patch to the skin further causes contact between the ingredients and the skin for skin sensitivity testing.

4. The cutaneous skin patch of claim 3, wherein the marked skin aids interpretation of results of the skin sensitivity testing.

5. The cutaneous skin patch of claim 1, wherein the ingredients are mixed with a base substance.

6. The cutaneous skin patch of claim 1, wherein each of the one or more wells comprises a foam frame that surrounds the ingredient and at least partially defines the well.

7. The cutaneous skin patch of claim 1, wherein the one or more landmarks cause the skin to be marked in a manner that indicates an orientation of the patch on the skin.

8. The cutaneous skin patch of claim 1, wherein the one or more landmarks cause the skin to be marked in a manner that indicates a location of at least one of the one or more wells.

9. The cutaneous skin patch of claim 1, further comprising:
at least one well containing a marking substance for marking the skin.

10. The cutaneous skin patch of claim 1, wherein the one or more landmarks include a stencil through which the skin is manually marked.

11. The cutaneous skin patch of claim 1, wherein the patch is applied to the skin by the user.

12. The cutaneous skin patch of claim 1, wherein the patch further comprises a removable liner that seals the one or more wells.

13. The cutaneous skin patch of claim 12, wherein one or more of the backing and the liner are waterproof or water-resistant.

14. The cutaneous skin patch of claim 12, further comprising a heat-resistant and moisture-resistant container surrounding the backing and the liner.

15. A method of manufacturing a cutaneous skin patch, comprising:
shaping a backing having an adhesive surface that adheres to skin of a human user;
configuring one or more wells on the backing;
incorporating one or more landmarks onto the backing; and
populating the one or more wells with ingredients mixed with a base.

16. The method of claim 15, further comprising:
sealing the patch with a waterproof or water-resistant liner.

17. The method of claim 15, further comprising:
printing on a reverse side of the backing one or more of:
instructions for using the patch;
an aesthetic design;
a brand;
a logo; and
a code.

18. The method of claim 15, wherein the one or more landmarks, when the patch is affixed to the skin, cause the skin to be marked to indicate an orientation of the patch.

19. The method of claim 15, wherein the one or more landmarks, when the patch is affixed to the skin, mark the skin to indicate locations of the one or more wells.

20. The method of claim 15, wherein the one or more landmarks include at least one of:
a temporary tattoo;
a stencil for manually marking the skin; and
means for transferring a semi-permanent marking substance to the skin.
